(19) United States
(12) Patent Application Publication
(10) Pub. No.: US 2025/0186199 A1
(43) Pub. Date: Jun. 12, 2025

(54) Methods for Reducing Leaflet Stress

(71) Applicant: St. Jude Medical, Cardiology Division, Inc., St. Paul, MN (US)

(72) Inventors: Brandon Moore, St. Louis Park, MN (US); Jay Reimer, Shoreview, MN (US); Michael Shane Morrissey, St. Paul, MN (US); Kent J. Smith, Shoreview, MN (US); Victoria Schuman, Minneapolis, MN (US); Tracee Eidenschink, Wayzata, MN (US); Sarah Tanck, Green Bay, WI (US)

(73) Assignee: St. Jude Medical, Cardiology Division, Inc., St. Paul, MN (US)

(21) Appl. No.: 18/928,284

(22) Filed: Oct. 28, 2024

Related U.S. Application Data
(60) Provisional application No. 63/607,235, filed on Dec. 7, 2023.

(51) Int. Cl. A61F 2/24; A61F 2/915

(52) U.S. Cl. CPC A61F 2/2418; A61F 2002/91583; A61F 2210/0057; A61F 2230/0013

(57) ABSTRACT

In some embodiments, a prosthetic heart valve includes a stent having a plurality of commissure attachment features, each of the plurality of commissure attachment features having an arch shape formed by a single continuous strut, a cuff coupled to the stent, a plurality of swatches, each of the plurality of swatches being coupled to each of the plurality of commissure attachment features, and a plurality of leaflets, the cuff and the plurality of leaflets forming a valve assembly, each of the plurality leaflets having two leaflet attachment regions, the leaflet attachment regions being coupled to a respective one of the plurality of swatches.

### CROSS-REFERENCE TO RELATED APPLICATIONS

This application claims priority to U.S. Provisional Patent Application No. 63/607,235, filed Dec. 7, 2023, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement (“TAVR”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to herein as a valve assembly) mounted to/within an expandable stent. In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets, and may also include a cuff or skirt. The cuff may be disposed on the stent's interior or luminal surface, its exterior or abluminal surface, and/or on both surfaces. A cuff helps to ensure that blood does not flow around the valve leaflets if the valve or valve assembly is not optimally seated in a valve annulus. A cuff, or a portion of a cuff, disposed on the exterior of the stent can help retard leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

When self-expandable prosthetic heart valves are delivered into a patient to replace a malfunctioning native heart valve, the self-expandable prosthetic heart valve is almost always maintained in the collapsed condition within a capsule of the delivery device. While the capsule may ensure that the prosthetic heart valve does not self-expand prematurely, the overlying capsule (with or without the help of additional internal retaining features) helps ensure that the prosthetic heart valve does not come into contact with any tissue prematurely, as well as helping to make sure that the prosthetic heart valve stays in the desired position and orientation relative to the delivery device during delivery. However, balloon expandable prosthetic heart valves are typically crimped onto the balloon of a delivery device without a separate capsule that overlies and/or protects the prosthetic heart valve. One reason for this is that space is always at a premium in transcatheter prosthetic heart valve delivery devices and systems, and adding a capsule in addition to the prosthetic valve and the underlying balloon may not be feasible given the size profile requirements of these procedures.

The continuous opening and closing of a prosthetic heart valve may result in mechanical stresses on the leaflet tissue (e.g., the leaflet attachment regions), which can affect performance and longevity of the valve. Specifically, the leaflet attachment region, which is coupled to the commissure attachment features may experience high stress during the repeated movement of the leaflets. Among other advantages, it would be beneficial to provide new prosthetic heart valve configurations with features for managing the high stresses in this region.

### BRIEF SUMMARY OF THE DISCLOSURE

In some embodiments, a prosthetic heart valve includes a stent having a plurality of commissure attachment features, each of the plurality of commissure attachment features having an arch shape formed by a single continuous strut, a cuff coupled to the stent, a plurality of swatches, each of the plurality of swatches being coupled to each of the plurality of commissure attachment features, and a plurality of leaflets, the cuff and the plurality of leaflets forming a valve assembly, each of the plurality leaflets having two leaflet attachment regions, the leaflet attachment regions being coupled to a respective one of the plurality of swatches.

In some embodiments, a prosthetic heart valve includes a stent having a plurality of commissure attachment features, a cuff coupled to the stent, a plurality of leaflets, the cuff and the plurality of leaflets forming a valve assembly, each of the plurality leaflets having two leaflet attachment regions, a free edge and a belly line, and a plurality of buffering elements independent of the plurality of leaflets, and being disposed along the belly line of each of the plurality of leaflets.

In some embodiments, a prosthetic heart valve includes a stent having a plurality of commissure attachment features, each of the plurality of commissure attachment features including a pair of flexible struts, a cuff coupled to the stent, and a plurality of leaflets, the cuff and the plurality of leaflets forming a valve assembly, each of the plurality leaflets having two leaflet attachment regions.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIGS. 2A-B illustrates a prosthetic heart valve PHV, crimped over a balloon in the deflated and inflated conditions.

FIG. 3 is a schematic illustration of an arched commissure attachment feature in a drum-like configuration.

FIGS. 4A-B illustrate a second embodiment showing the attachment of a leaflet to an elastic strain relief.

FIGS. 5A-B illustrate a third embodiment showing the attachment of a leaflet to a pledged pad.

FIG. 6 illustrates a fourth embodiment showing the attachment of a leaflet to a commissure attachment feature and damper struts.

FIGS. 7-8B illustrate a fifth embodiment showing the attachment of a leaflet to a commissure attachment feature having loops and/or cantilevered arms.

### DETAILED DESCRIPTION

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation, but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to a trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

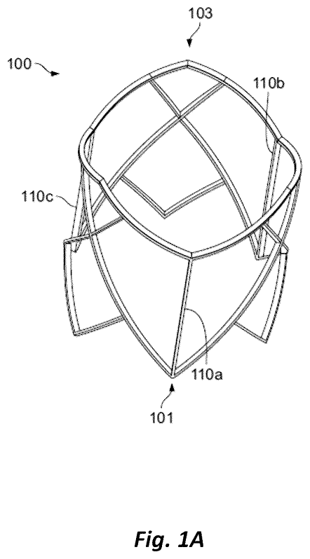

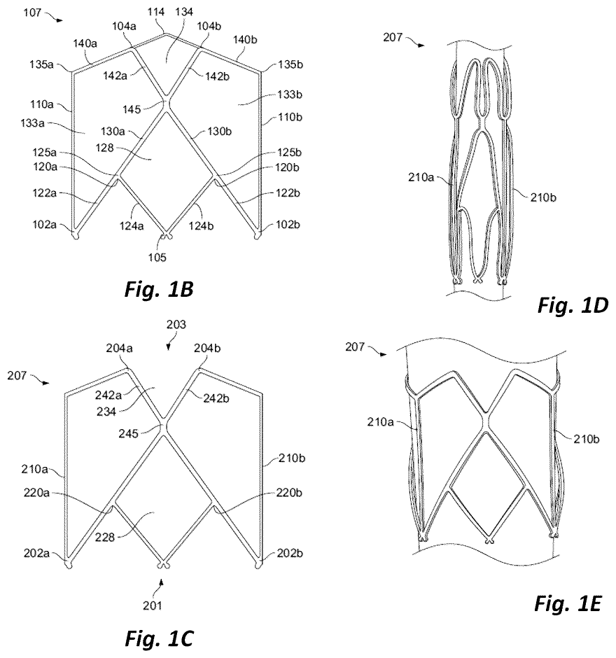

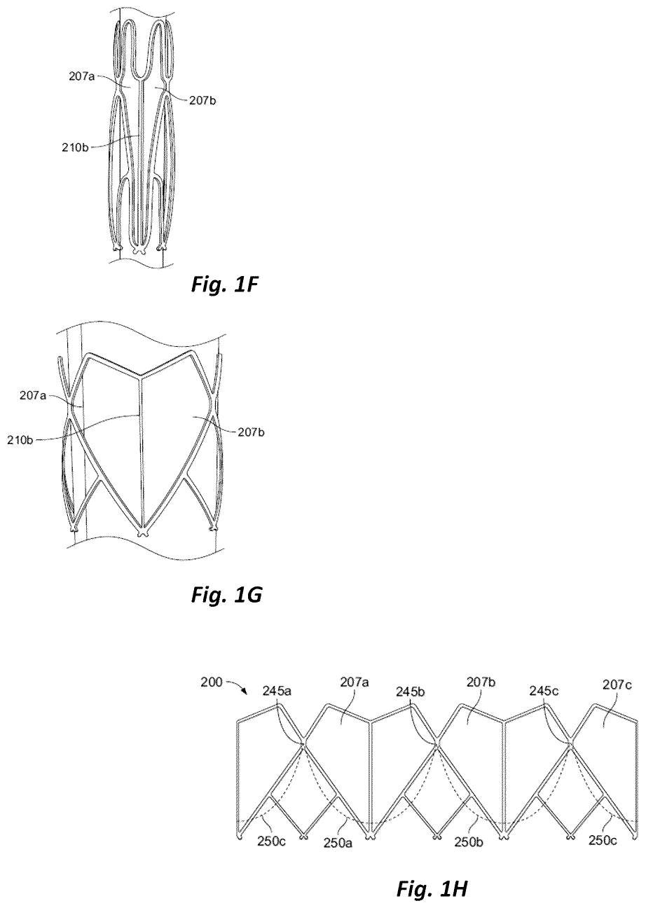

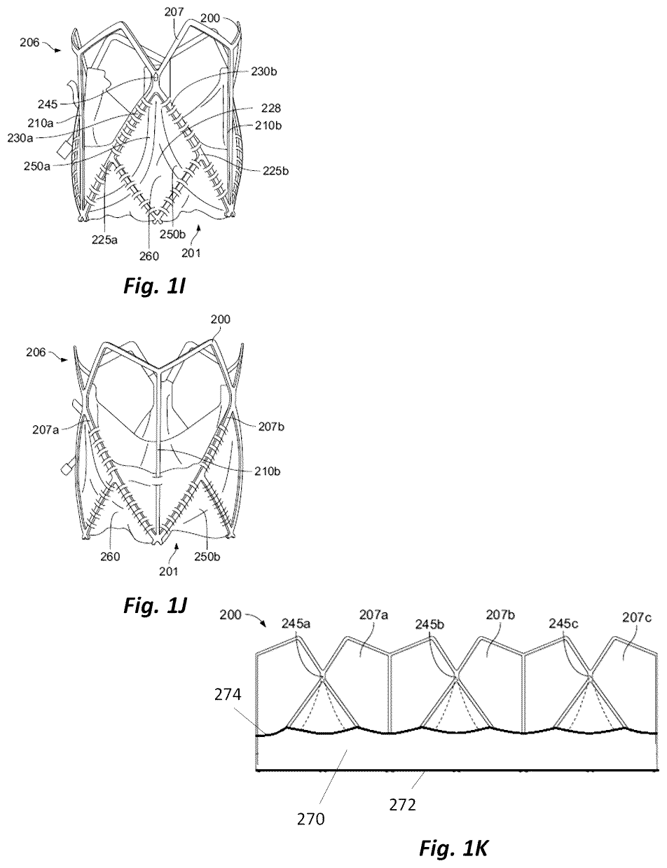

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100, and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135a and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage). In the embodiment illustrated in FIGS. 1I-J, the cuff 260 only covers about half of the stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g., wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example including transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstance, under an overlying sheath). Upon arrival at or adjacent the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

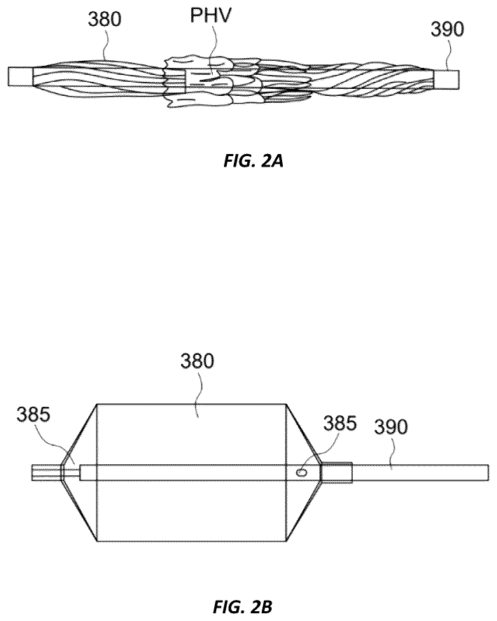

Referring to FIG. 2A, an example of a prosthetic heart valve PHV, which may include a stent similar to stents 100 or 200, is shown crimped over a balloon 380 of a balloon catheter 390 while the balloon 380 is in a deflated condition. It should be understood that other components of the delivery device, such as a handle used for steering and/or deployment, as well as a syringe for inflating the balloon 380, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 390 to inflate the balloon 380, as shown in FIG. 2B. FIG. 2B omits the prosthetic heart valve PHV, but it should be understood that, as the balloon 380 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 380 through a lumen within balloon catheter 390 and into one or more ports 385 located internal to the balloon 380. In the particular illustrated example of FIG. 2B, a first port 385 may be one or more apertures in a side wall of the balloon catheter 390, and a second port 385 may be the distal open end of the balloon catheter 390, which may terminate within the interior space of the balloon 380.

The present disclosure provides several techniques, devices and methods to reduce stresses in a leaflet material by allowing reduction, redistribution and/or dampening of stresses during valve operation. The devices, methods and techniques provided herein relate to unique attachment methods between the leaflets, cuffs, stents, buffers, and/or commissure attachment features adjacent high stress regions of the valve. Additionally, one goal is to reduce stress while maintaining other performance metrics (e.g., coaptation, motion, etc.). Certain design principles disclosed herein aim to add deflection and/or flexibility at the leaflet attachment point to offload stresses from the leaflet. While several embodiments are shown, it will be appreciated that certain features may be combined with one another.

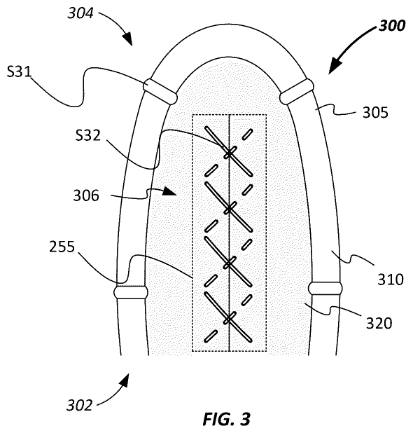

FIG. 3 shows a first commissure attachment feature 300 for coupling to one or more leaflet attachment regions 255 of leaflets 250. Commissure attachment feature 300 may extend between a proximal end 302 and a distal end 304 and include a generally arched or rounded body 305. In some examples, the commissure attachment features 300 are disposed in the distalmost position on the stent (e.g., no other stent element extends distally beyond the commissure attachment feature). In this example, rounded body 305 includes a single continuous arched strut 310 that defines a central window 306. Strut 310 may be coupled to, or unitarily formed with, a remainder of a stent at proximal end 302 and may be unattached (e.g., cantilevered) at distal end 304. In some examples, a complementary-shaped (e.g., arched, rounded, or substantially rectangular) swatch 320 may be placed on the luminal or abluminal surface of the commissure attachment feature 300 and attached thereto with sutures S31 or other appropriate coupling means. Swatch 320 be formed of fabric, or any of the materials of the leaflets or cuff (e.g., a biological material, such as animal pericardium, or synthetic materials, such as ultra-high molecular weight polyethylene). A second suture pattern S32 may travel in and out of swatch 320 and attach it to one or more leaflet attachment region(s) 255 as shown. In one example, second suture pattern S32 may include a single column of sutures as shown. Notably, leaflet attachment regions 255 may be only directly coupled to swatch 320, and not directly to the commissure attachment feature 300 or any other stent element to add compliance at the commissure and produce a “drum” or “trampoline” effect. Additionally, the shape of commissure attachment feature 300 may eliminate the possibility for stress concentration in the corners and lead to a generally more uniform arrangement that distributes stress over the swatch surface, while allowing flexion of the commissure attachment feature.

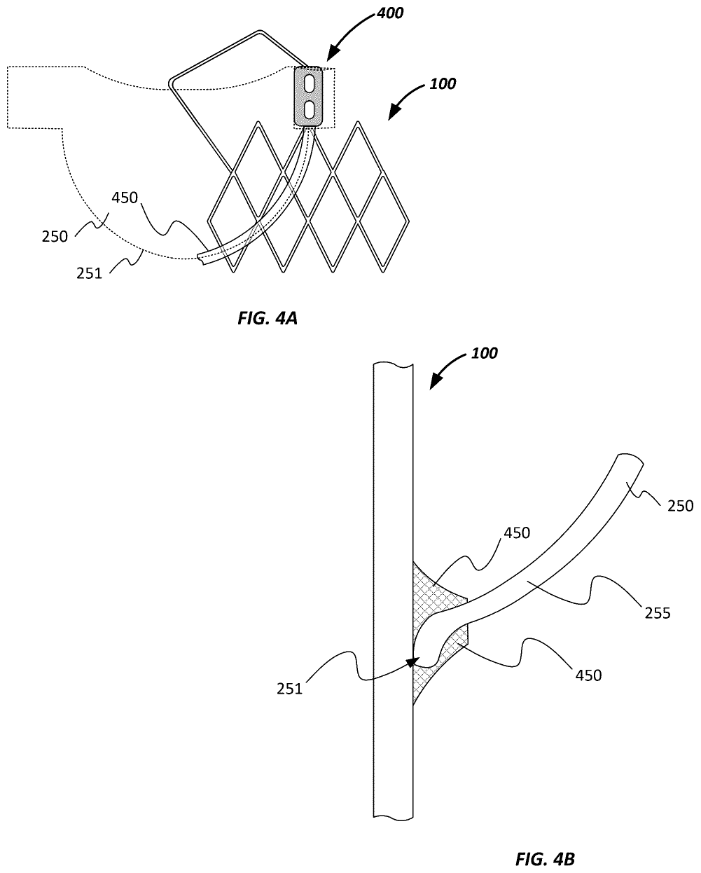

FIG. 4A illustrates an example of a stent 100 having a commissure attachment feature 400 and an elastic strain relief member 450 coupled to the stent and/or cuff. In this example, elastic strain relief member 450 may include a U-shaped compressible material that extends from one commissure attachment feature to and adjacent commissure attachment feature. In some examples, the elastic relief member 450 may deflect up to 100 μm. In some examples, the compressible material has characteristics that mimic cartilage. In some examples, the elastic relief member 450 may be configured to stretch about 0.1 mm or more when coupled. Elastic strain relief member 450 may be formed of fabric, or any of the materials of the leaflets or cuff (e.g., a biological material, such as animal pericardium, or synthetic materials, such as ultra-high molecular weight polyethylene, silicone, hydrogels, woven, knit or electrospun synthetic materials, or foam). In some examples, elastic strain relief member 450 may be incorporated along suture lines that attach the stent to leaflet belly 251 of leaflet 250. Elastic strain relief member 450 may be coupled to the stent struts (FIG. 4B) or to the cuff, or to both and may form a contoured line along the full length or a partial length of leaflet belly 251. As shown, elastic strain relief member 450 may be disposed above and/or below leaflet belly 251 to buffer the leaflet material during opening and/or closing so that the leaflet does not hit the cuff and/or stent and abrade. In some examples, elastic strain relief member may provide strain relief during valve closing, and forces imparted on a leaflet from blood pressure may be translated to a compliant material to avoid a sudden point load at suture points.

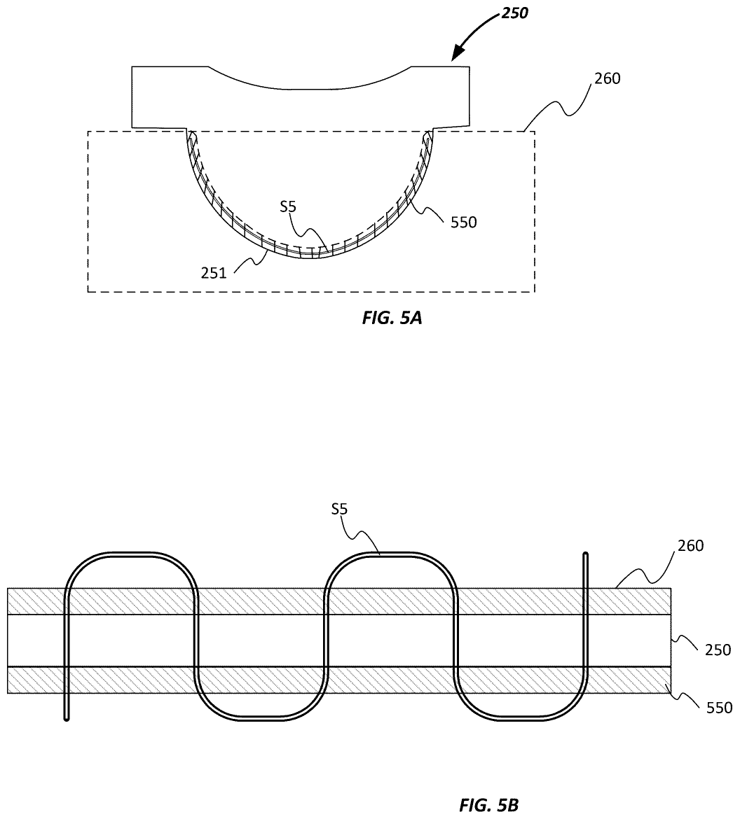

FIGS. 5A-B illustrate another example of a pledget-style configuration for coupling a leaflet belly line 251 to a cuff 260. In this example, a U-shaped pad 550 is disposed opposite cuff 260 and used to buffer the suture line at the attachment points to the frame and/or fabric. In some examples, pad 550 comprises a generally flat structure formed of a biological material, such as animal pericardium, or synthetic materials, such as ultra-high molecular weight polyethylene or foam or fabric. The added pad may act as a reinforcement material that prevents the suture from digging into the leaflet material and may reduce stress concentration by squishing the leaflet at times of high stress. As shown, pledgets in the form of small cotton and/or fabric pads may help keep sutures from ripping through tissue during procedures. Pledget pad 550 may be disposed radially inward or outward of inner cuff 260 and may create a sandwich around leaflet 250. Alternatively, a single layer may be used (inside or outward of inner cuff 260. A suture S5 may be used to couple any combination of pad 550, leaflet 250 and cuff 260. In some examples, suture S5 may travel in and out of the three layers as shown in FIG. 5B.

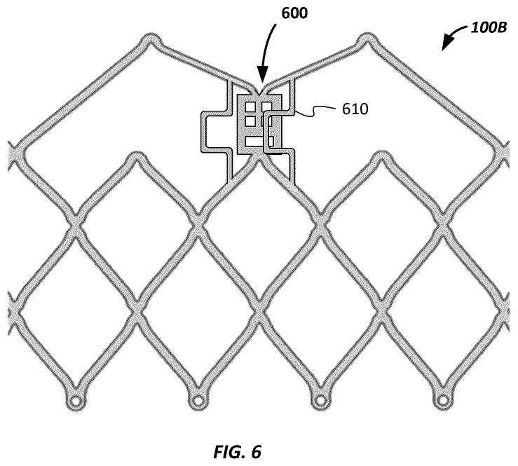

The stent may also be modified to include features that reduce stress on the leaflets (e.g., the leaflet attachment portions). FIG. 6 illustrates a stent 100B that includes a rigid or semi-rigid commissure attachment feature 600. One or more leaflets may be attached to the stent at, or through, commissure attachment feature 600. In addition to, or instead of, commissure attachment feature 600, one or more leaflets may be attached to damper struts 610 (e.g., by folding over it, wrapping around it, directly or indirectly being coupled to it, or suturing through or around it if the damper strut 610 includes eyelets). That is, the leaflets may be attached to the damper struts 610 only, the commissure attachment feature 600 only, or to both. In one example, sutures may travel through eyelets of commissure attachment feature 600, which functions as an attachment guide, and attach to one or more leaflets to damper struts 610. A buffering or spacing material (e.g., similar to swatch 320) may be disposed between commissure attachment feature 600 and damper struts 610. In some examples, damper struts 610 are more flexible than the remaining struts and/or the commissure features. For example, damper struts 610 may have a thinner cross-section or thickness than remaining struts of the stent (e.g., ¼, ⅓, ½, or ⅔ of the cross-section or thickness of other struts). Alternatively, or additionally, damper struts 610 may have a different material than the remaining struts of the stent (e.g., materials with a lower elastic modulus than other materials of the stent) and allow for increased motion during load and/or unloading of the leaflets. In some examples, a secondary operation may be applied to the damper struts 610 (e.g., a mechanical, chemical, or other operation or treatment) to make them more flexible. In some examples, damper struts 610 are Damper struts 610 may be biased corresponding to an open or closed leaflet position in order to tailor the amount of resistance or commissure deflection.

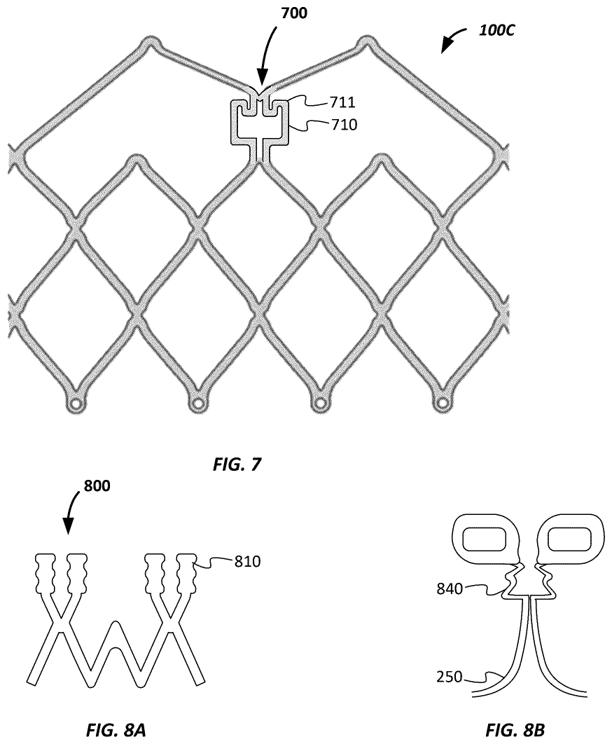

FIG. 7 illustrates a stent 100C that includes a cantilever commissure design. One or more leaflets may be attached to the stent at commissure attachment feature 700. In this example, commissure attachment feature 700 has two opposing struts 710 that act as cantilevered arms, and each of the struts 710 includes an upper loop 711. When leaflets are attached to struts 710 they may flex during loading, reducing stress in certain regions. It will be understood that the arm length and dimensions of struts 710 may be modified as desired to control deflection magnitude. In the example shown, struts 710 face one another and form a mirror-image arrangement referred to as a “cactus shape”. In one variation, shown in FIG. 8A, a commissure attachment feature 800 includes two cantilevered struts 810 that are unattached at the top. In FIG. 8B, leaflet tissue may be provided in an accordion state (e.g., with bunched/gathered/folded or accordion portions 840) near the commissure so that it can unfold when stress is applied. The folded tissue would need some shape memory to return to its original position when stress is released, which could be accomplished through suturing or potentially by fixing the tissue in a folded configuration. The objective of this embodiment is to alleviate stress by “unfolding” rather than purely stretching the leaflet material.

In use, a prosthetic heart valve may be crimped, loaded and delivered into position, the prosthetic valve having elements that serve to distribute stress and increase compliance. These features may be used to isolate the leaflets, or tabs of the leaflets, and reduce the stress on the valve components during use. It is to be understood that the embodiments described herein are merely illustrative of the principles and applications of the present disclosure. For example, a system may combine suturing techniques, commissure attachment features, leaflet attachment regions and/or buffering materials from different embodiments. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve, comprising:
a stent having a plurality of commissure attachment features, each of the plurality of commissure attachment features having an arch shape formed by a single continuous strut;
a cuff coupled to the stent;
a plurality of swatches, each of the plurality of swatches being coupled to each of the plurality of commissure attachment features; and
a plurality of leaflets, the cuff and the plurality of leaflets forming a valve assembly, each of the plurality leaflets having two leaflet attachment regions, the leaflet attachment regions being coupled to a respective one of the plurality of swatches.

2. The prosthetic heart valve of claim 1, wherein the leaflet attachment regions are not coupled directly to the single continuous strut.

3. The prosthetic heart valve of claim 1, wherein each of the plurality of commissure attachment features is coupled to the stent a proximal end an unattached at a distal end.

4. The prosthetic heart valve of claim 1, wherein each of the plurality of commissure attachment features is disposed at a distalmost position on the stent.

5. The prosthetic heart valve of claim 1, wherein the plurality of plurality of swatches comprises a fabric.

6. The prosthetic heart valve of claim 1, wherein the plurality of plurality of swatches comprises a biological material.

7. A prosthetic heart valve, comprising:
a stent having a plurality of commissure attachment features;
a cuff coupled to the stent;
a plurality of leaflets, the cuff and the plurality of leaflets forming a valve assembly, each of the plurality of leaflets having two leaflet attachment regions, a free edge and a belly line; and
a plurality of buffering elements independent of the plurality of leaflets, and being disposed along the belly line of each of the plurality of leaflets.

8. The prosthetic heart valve of claim 7, wherein the plurality of buffering elements comprises a U-shaped elastic straight relief member.

9. The prosthetic heart valve of claim 8, wherein the U-shaped elastic straight relief member comprises a compressible material.

10. The prosthetic heart valve of claim 8, wherein the U-shaped elastic straight relief member is disposed above and below the belly line of a respective one of the plurality of leaflets.

11. The prosthetic heart valve of claim 7, wherein the plurality of buffering elements comprises flat pledged U-shaped pads.

12. The prosthetic heart valve of claim 11, wherein the flat pledged U-shaped pads may be disposed opposite the cuff to sandwich each of the plurality of leaflets.

13. The prosthetic heart valve of claim 12, further comprising a suture that pierces through at least ones of the flat pledged U-shaped pads, the plurality of leaflets and the cuff.

14. The prosthetic heart valve of claim 13, wherein the suture pierces through all of the flat pledged U-shaped pads, the plurality of leaflets and the cuff.

15. A prosthetic heart valve, comprising:
a stent having a plurality of commissure attachment features, each of the plurality of commissure attachment features including a pair of flexible struts;
a cuff coupled to the stent; and
a plurality of leaflets, the cuff and the plurality of leaflets forming a valve assembly, each of the plurality leaflets having two leaflet attachment regions.

16. The prosthetic heart valve of claim 15, wherein each commissure attachment feature includes a generally rectangular commissure attachment feature having eyelets, and the pair of flexible struts include damper struts that are more flexible than other struts of the stent.

17. The prosthetic heart valve of claim 16, wherein the plurality of leaflets are coupled to the pair or flexible struts with a suture that passes through eyelets of the commissure attachment features.

18. The prosthetic heart valve of claim 17, further comprising buffering materials disposed between each pair of flexible struts and the corresponding rectangular commissure attachment feature.
